(19) United States
(12) Patent Application Publication
(10) Pub. No.: US 2015/0032103 A1
(43) Pub. Date: Jan. 29, 2015

(54) Bipolar Ablation Device

(71) Applicant: Cook Medical Technologies LLC, Bloomington, IN (US)

(72) Inventors: Tyler E. MCLAWHORN, Winston-Salem, NC (US); Vihar C. SURTI, Winston-Salem, NC (US)

(21) Appl. No.: 14/341,517

(22) Filed: Jul. 25, 2014

Related U.S. Application Data
(60) Provisional application No. 61/859,365, filed on Jul. 29, 2013.

(51) Int. Cl. A61B 18/14

(52) U.S. Cl. CPC A61B 18/1492; A61B 2018/00166
USPC 606/41

(57) ABSTRACT

A bipolar ablation device and methods for using the same. The bipolar ablation device has a first catheter with a first expandable electrode and a second catheter with a second expandable electrode. The first expandable electrode is expanded on a first side of a septum between two chambers and the second expandable electrode is expanded on a second side of the septum. The size of the electrodes relative to one another is adjusted to concentrate the ablation on one side of the septum and electrical energy is delivered to the electrodes to ablate the tissue.

### RELATED APPLICATIONS

The present patent document claims the benefit of the filing date under 35 U.S.C. §119(e) of Provisional U.S. Patent Application Ser. No. 61/859,365, filed Jul. 29, 2013, which is hereby incorporated by reference.

### FIELD

This invention relates generally to medical devices for ablating tissue. More particularly, this invention relates to a system for ablating tissue across a wall, such as a septum between chambers of the heart.

BACKGROUND

Hypertrophic obstructive cardiomyopathy (HOCM) is one particular disease that may benefit from RF ablation if the RF energy were able to reach the appropriate depth. Hypertrophic obstructive cardiomyopathy (HOCM) is a disease of the myocardial tissue where the heart tissue thickens. It can oftentimes obstruct the outflow tract of blood flow from the left ventricle to the aorta. Current treatment of HOCM includes a surgical myectomy in which excision of a portion of the basal septum eliminates the obstructive area. Results of this procedure are fairly effective; however the procedure is an open surgery which has its own inherent risks and complications.

A more minimally invasive approach is alcohol ablation via an intravascular approach. For this procedure, a physician will balloon occlude a branch of the septal perforator arteries (which supply blood to the ventricular septum) and inject ethanol to the area downstream of the occlusion. The ethanol causes localized myocardial infarction and eventual elimination of the obstructive area. However, this approach is limited by the size and location of the septal perforator arteries. The physician also risks spillage of the ethanol behind the balloon occlusion which can cause necrosis to untargeted tissue. Additionally, the physician is at the mercy of the pathway of the vessels and risks crossing the pathway of the Bundle of His which would affect the electrical conduction of the ventricle potentially resulting in permanent pacemaker implantation.

Recently, attempts to treat this disease via radio frequency (RF) ablation have been attempted. RF ablation is a medical procedure in which live tissue is scarred or destroyed, often to disrupt electrical signals in the body. Currently, there are multiple cardiac conditions that may be treated using radio frequency (RF) ablation to either disrupt the electrical signaling pathway or to ‘remodel’ cardiac tissue. Oftentimes, to be effective, the RF energy must penetrate deep into the target tissue to have the desired clinical effect. Deep penetration of RF energy may be difficult to achieve and current techniques often cannot reach the appropriate depth.

To date, ablation device for treating HOCM have not been able to achieve the necessary depth of ablation required to clinically relieve the outflow obstruction. These current attempts have used ablation devices have relatively small surface area and send energy from an active electrode on a distal tip of the device to a neutral electrode applied externally to the patient's skin. In order to increase the depth of ablation, the energy supplied to the active electrode can be increased. However, increases of energy are transmitted through the body to the patient's skin which may cause complications.

It would be beneficial to have an ablation system capable of penetrating deep into tissue without the risk associated with RF energy travelling through the body.

SUMMARY

Embodiments of the invention include an ablation device comprising a first elongated member and a second elongated member. The first elongated member has a first distal end and a second proximal end. A first electrode is disposed at the first distal end of the first elongated member and is in electrical communication with the first proximal end of the first elongated member. The first electrode is expandable from a collapsed configuration to an expanded configuration having a first size. The second elongated member has a second distal end and a second proximal end. A second electrode is disposed at the second distal end of the second elongated member and is in electrical communication with the second proximal end of the second elongated member. The second electrode is expandable from a collapsed configuration to an expanded configuration having a second size larger than the first size.

Embodiments of the invention further include a method of ablating a septum utilizing a bipolar ablation device having a first electrode in electrical communication with a first pole of a power source and a second electrode in electrical communication with a second pole of a power source. In the method the first electrode is guided to an ablation target area on a first side of a septum. The first electrode is then expanded to an expanded configuration having a first size. The second electrode is guided to an area on a second side of the septum. The second electrode is expanded to an expanded configuration having a second size greater than the first size. Power is provided from the power source to the first electrode and the second electrode to ablate tissue at the target area.

In another embodiment, an ablation device includes a first ablation assembly and a second ablation assembly. The first ablation assembly comprises a first outer catheter, a first inner catheter, a first flexible mesh, and first flexible conductive coating, and a first conductor. The first outer catheter has a first outer distal end and a first outer proximal end. The first inner catheter has a first inner distal end and a first inner proximal end. The first inner catheter is slidably disposed within a lumen of the first outer catheter. The first flexible mesh has a first flexible mesh proximal end coupled to the first outer distal end and a first flexible mesh distal end coupled to the first inner distal. The first flexible conductive coating is disposed on the first flexible mesh and the first conductor is configured to couple the first flexible conductive coating to a first pole of a power source. The second ablation assembly comprises a second outer catheter, a second inner catheter, a second flexible mesh, a second flexible coating, and a second conductor. The second outer catheter has a second outer distal end and a second outer proximal end. The second inner catheter has a second inner distal end and a second inner proximal end. The second inner catheter is slidably disposed within a lumen of the second outer catheter. The second flexible mesh has a second flexible mesh proximal end coupled to the second outer distal end and a second flexible mesh distal end coupled to the second inner distal. The second flexible conductive coating is disposed on the second flexible mesh and the conductor is configured to couple the second flexible conductive coating to a second pole of a power source.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 illustrates a cross section of a healthy heart.

FIG. 2 illustrates a cross section of a heart experiencing HOCM and having an embodiment of a bipolar ablation device disposed therein.

FIG. 3 illustrates a distal end of a catheter assembly for use in a bipolar ablation device.

FIG. 4 illustrates the distal end of the catheter assembly of FIG. 3 with an electrode in an expanded configuration.

FIG. 5 illustrates a bipolar ablation device ablating a septum.

FIG. 6 illustrates a distal end of an alternative catheter assembly for use in a bipolar ablation device.

FIG. 7 illustrates the alternative catheter assembly of FIG. 6 with the electrode in an expanded configuration.

FIG. 8 illustrates another embodiment of a bipolar ablation device assembly.

FIG. 9 illustrates another embodiment of a bipolar ablation device assembly.

The drawings are not necessarily to scale.

### DETAILED DESCRIPTION

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Detailed Description does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

In the following discussion, the terms “proximal” and “distal” will be used to describe the opposing axial ends of the inventive ablation device, as well as the axial ends of various component features. The term “proximal” is used in its conventional sense to refer to the end of the ablation device (or component thereof) that is closest to the operator during use of the ablation device. The term “distal” is used in its conventional sense to refer to the end of the ablation device (or component thereof) that is initially inserted into the patient, or that is closest to the patient during use. For example, an ablation device may have a proximal end and a distal end, with the proximal end designating the end closest to the operator, such as a handle, and the distal end designating an opposite end of the ablation device. Similarly, the term “proximally” refers to a direction that is generally towards the operator along the path of the ablation device and the term “distally” refers to a direction that is generally away from the operator along the ablation device.

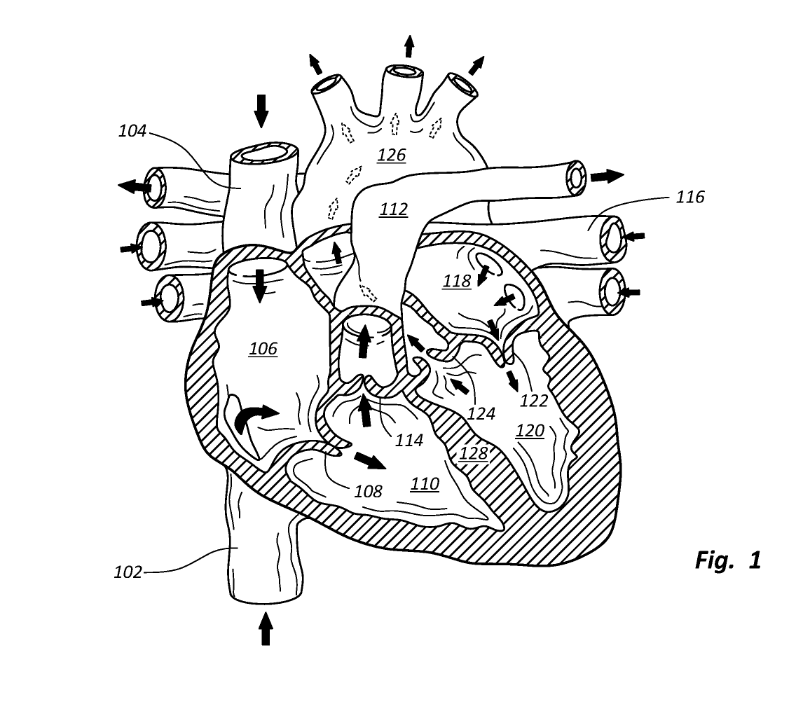

FIG. 1 is a simplified cut-away view of a healthy heart 100 showing the various chambers and vessels of the heart 100. Blood (depicted by arrows) flows to the heart 100 from the body through the inferior vena cava 102 and the superior vena cava 104 into the right atrium 106. From the right atrium 106 blood flows through the tricuspid valve 108 into the right ventricle 110. From the right ventricle 110 blood is pumped into the pulmonary artery 112 through the pulmonary valve 114. The blood flows into the lungs from the pulmonary artery 112 and returns via the pulmonary vein 116. From the pulmonary vein 116, blood collects in the left atrium 118 and flows into the left ventricle 120 through the mitral valve 122. The right ventricle 110 and the left ventricle 120 are separated by a septum 128. Blood flows from the left ventricle 120 through the aortic valve 124 to the aorta 126 where it is delivered to the body.

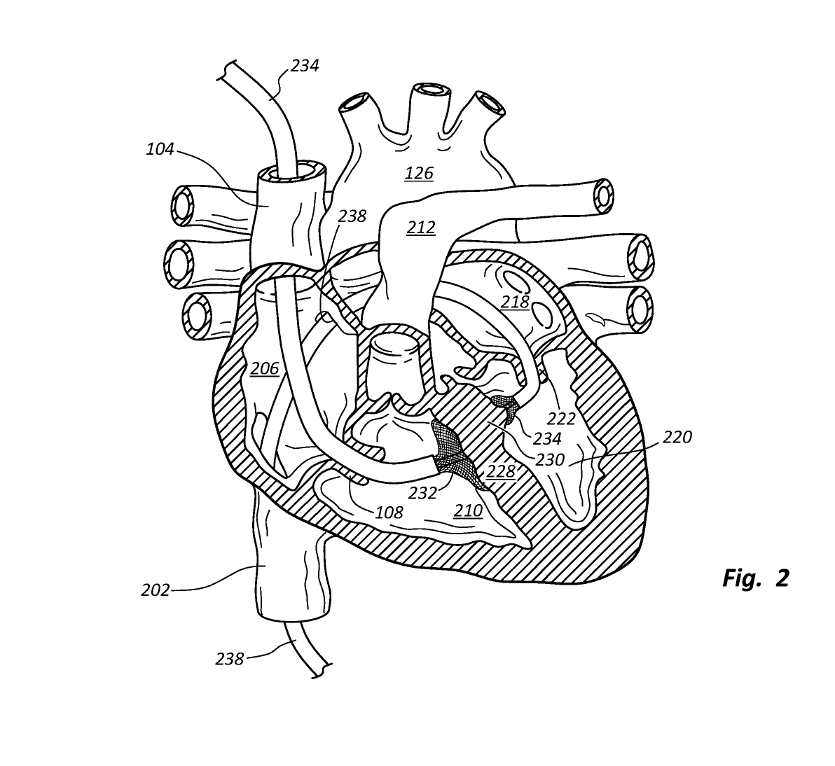

FIG. 2 is a simplified cut away view of a heart 200 in an advanced stage of HOCM.

The septum 228 is thicker than the septum 128 of the healthy heart of FIG. 1 and the basal septum 230 partially blocks the aortic valve 224. Treatment for HOCM includes removing tissue from the thickened basal septum 230, thereby restoring blood flow from the left ventricle 220 to the aorta 226. Care must be taken to remove tissue primarily on the left ventricle 220 side of the septum 228 to avoid perforation of the septum 228.

An embodiment of a bipolar ablation device is disposed within the heart 200 of FIG. 2. The bipolar ablation device has a first electrode 232 positioned in the right ventricle 210 and a second electrode 234 positioned in the left ventricle 220. The basal septum 230 is ablated by passing electrical energy between first electrode 232 and the second electrode 234 in a bipolar manner.

In the embodiment of FIG. 2, the first electrode 232 is introduced into the right ventricle 210 by a first catheter 234 extending through the tricuspid valve 108 by way of the superior vena cava 104. The second electrode 234 is introduced into the left ventricle 220 by way a second catheter 236 that extends through a perforation 238 in the atrial septum (not shown) between the right atrium 206 and the left atrium 218. The second electrode 234 passes from the inferior vena cava 202 into the right atrium 206 and then through the atrial septum into the left atrium 218. Finally, the second electrode 234 passes from the left atrium 218 into the left ventricle 220 through the mitral valve 222. Other techniques may be used to introduce the electrodes into the ventricles. Available options include trans-vascular, trans-apical, through open surgery, or other combinations of access techniques.

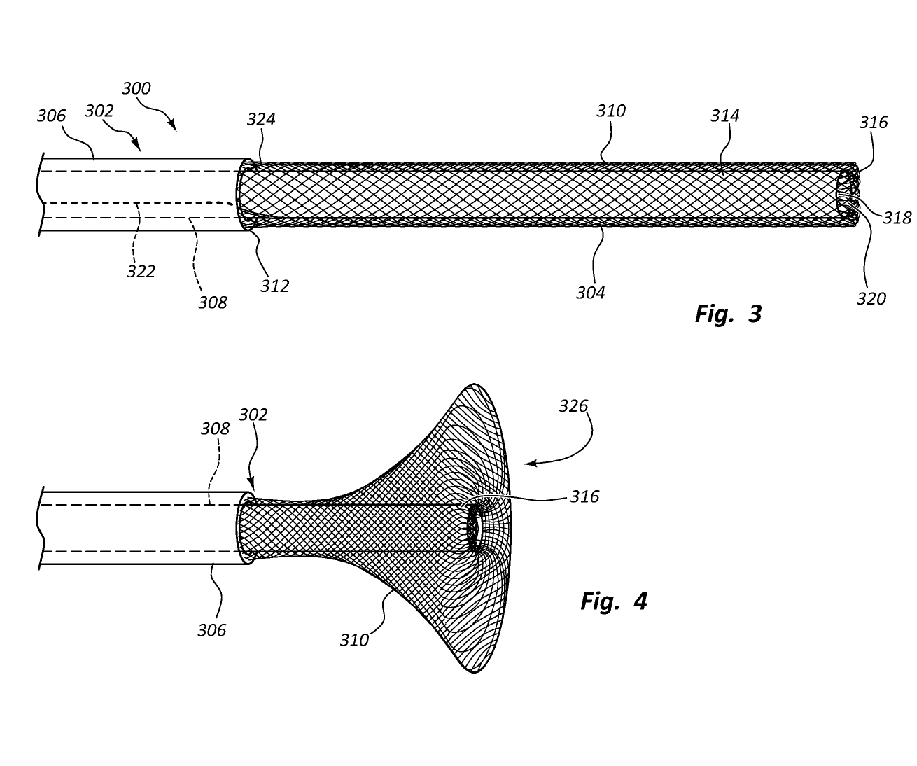

FIG. 3 illustrates a distal end 302 of an elongated member suitable for use in the present invention. The elongated member of FIG. 3 comprises a catheter assembly 300 having an electrode 304 suitable for use in the present invention. The catheter assembly 300 includes an outer catheter 306, an inner catheter 308, and a mesh member 310. The inner catheter 308 may be coaxially positioned within the outer catheter 306 and slidably positionable relative to the outer catheter 306. The outer catheter 306 includes a proximal end portion (not shown), a distal end portion 302 and a lumen 312 extending at least partially therethrough.

The mesh member 304 is has a conductive surface that acts as an electrode. In some embodiments, only points of the surface may be conductive such that the electrode acts as a multipoint source. The mesh member 304 is operably connectable to the inner catheter 308 and the outer catheter 306. As the inner catheter 308 and the outer catheter 306 are moved relative to each other, the shape of the mesh member 310 changes. In some embodiments, a distal end portion 314 of the mesh member 310 may be extended over a distal end 316 of the inner catheter 308, inverted into a lumen 318 of the inner catheter 308, and operably connected to an inner surface 320 of the inner catheter 314. A conductor 322 is configured to transmit current from a power source to the mesh member 310 and to the tissue (described in more detail below). A proximal end portion 324 of the mesh member 310 may be operably connected to the distal end portion 302 of the outer catheter 306.

FIG. 3 illustrates an extended configuration of the catheter assembly 300 where the distal end 316 of the inner catheter 308 is extended distal relative to the distal end portion 302 of the outer catheter 306 and the mesh member 310 is fully extended so that the mesh member 310 has an outer diameter that is about the same as an outer diameter of the outer catheter 306. The mesh member 310 expands, extends, and retracts by longitudinal movement of the inner catheter 308 relative to the outer catheter 306. The electrode may be delivered to the treatment site with the catheter assembly 300 in the extended configuration shown in FIG. 3. An outer sheath may be positioned over the distal end of the catheter assembly 300 for delivery to a treatment site.

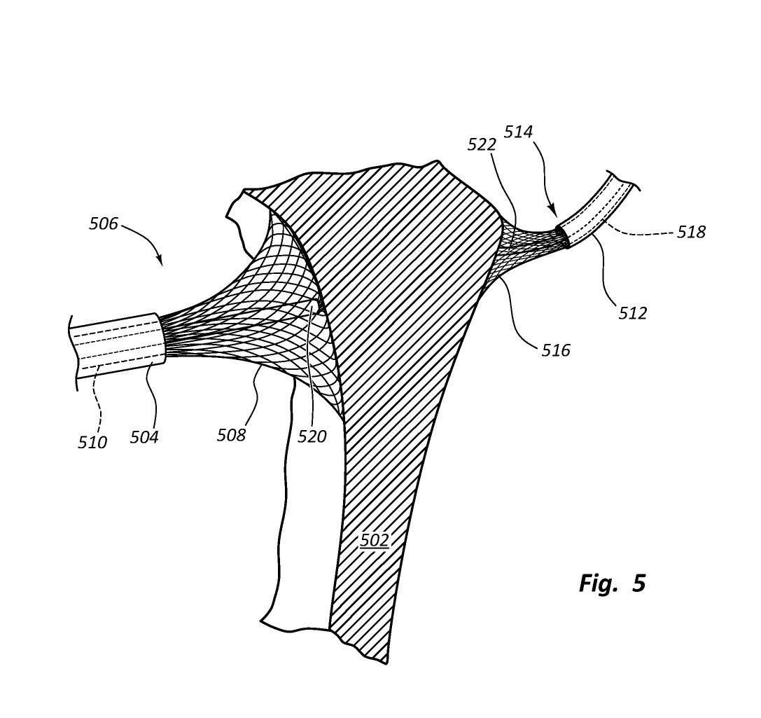

FIG. 4 illustrates the catheter assembly 300 in an expanded configuration. The inner catheter 308 is shown in FIG. 4 proximally withdrawn relative to the position of the inner catheter 308 shown in FIG. 3 axially compressing the mesh member. In some embodiments the compression may be provided by another means such as retracting the conductor 322 relative to the outer catheter 306. The distal end 316 of the inner catheter 308 is still distal to but closer to the distal end 302 of the outer catheter 306. As shown in FIG. 4, with the inner catheter 308 proximally withdrawn relative to the outer catheter 306 and still having the distal end 316 distal to the distal end portion 302 of the outer catheter 306, the mesh member 310 is radially expanded relative to the extended configuration and has an outer diameter greater than the outer diameter of the outer catheter 306. The mesh member 310 can be expanded a variable amount dependent upon the relative move movement of the two catheters. As shown in FIG. 4, the inner catheter 308 can be withdrawn to the point where an end face 326 of the mesh member 310 forms a generally flattened surface that can be advanced into contact with the tissue at the treatment site.

The mesh member 310 may be formed from wire such as nickel titanium alloys, for example, Nitinol, stainless steel, cobalt alloys, and titanium alloys. In some embodiments, the mesh may be formed from a polymeric material such as a polyolefin, a fluoropolymer, a polyester, for example, polypropylene, polytetrafluoroethylene, polyvinylidene fluoride, polyethylene terephthalate (PET), and combinations thereof. Other materials known to one skilled in the art may also be used to form the mesh member 310. In some embodiments the mesh member may comprises a combination of conductive and non-conductive materials.

In embodiments in which the mesh member 310 is formed of a non-conductive material, the mesh member 310 may be coated with a conductive material to form an electrode surface. For example, conductive ink may be applied to the exterior of the mesh member 310. The conductive ink may be applied in any pattern and spacing to be used for tissue treatment. In some embodiments, the conductive ink may be a silver-based ink. An exemplary silver-based ink may be obtained from Conductive Compounds (product number AG-510, Hudson, N.H.). However, other types of conductive ink may also be used, such as platinum-based, gold-based, and copper-based inks The inks may be epoxy-based inks or non-epoxy inks, such as urethane inks In some embodiments, the active portions of the mesh member 310 may comprise conductive polymers. The conductive ink may be applied to the mesh member 310 with a variety of printing processes, such as pad printing, ink jet printing, spraying, marker striping, painting, or other like processes. In some embodiments, the conductive ink may be applied to the mesh member with by spraying, dipping, painting or an electrostatic coating process.

FIG. 5 illustrates a bipolar ablation device 500 being deployed on a septum 502, such as the basal septum of the heart. The bipolar ablation device comprises a first catheter 504 and a second catheter 506. While the bipolar ablation device is shown ablating a septum between two chambers, embodiments of the invention are not limited to such. For example, the bipolar ablation device 500 could be used to ablate tissue on a wall of a heart between an inner chamber and an exterior of the heart. In such an embodiment one electrode would be guided to treatment site within the heart, and a second electrode would be placed on the exterior of the heart.

The first catheter 504 has a distal end 506 and a proximal end (not shown). The distal end 506 has an electrode 508 disposed thereon that is expandable from a collapsed configuration shown in FIG. 3 to an expanded configuration shown in FIG. 4 and FIG. 5. The proximal end of the first catheter 504 is electrically coupled to a first pole of a power source. A conductor 510 extends from the proximal end of the first catheter 504 to the electrode 508 to provide electrical communication to the electrode 508.

The second catheter 512 has a distal end 514 and a proximal end (not shown). The distal end 514 has an electrode 516 disposed thereon that is expandable from a collapsed configuration shown in FIG. 3 to an expanded configuration shown in FIG. 4 and FIG. 5. The proximal end of the second catheter 512 is electrically coupled to a second pole of a power source. A conductor 518 extends from the proximal end of the catheter to the electrode 516 to provide electrical communication to the electrode 516.

The first electrode 508 is expanded to have a larger end face 520 than an end face 522 of the second electrode 516. When power is applied to the electrodes 508, 516 from the poles of the power source, a circuit is formed between the two electrodes 508, 516. The circuit ablates the tissues between the electrodes 508, 516. The ablation may be biased from one side or the other by adjusting the size of the electrodes 508, 516 relative to one another. For example, in FIG. 5, having the first electrode 508 larger than the second electrode 516 results in the current density being largest at the second electrode 516. As such, any tissue effect resulting from the electrodes 508, 516 will be biased towards the second electrode 516 and away from the first electrode 508. Thus, the smaller electrode will be the ‘active’ electrode while the larger one would act as a ‘return’ electrode. In some embodiments, only a single electrode may be expandable. For example, a conventional electrode may be used as the smaller, active electrode, and a larger, expandable electrode used as the return electrode.

In the case of HOCM, it is advantageous to ablate the ventricular septum in such a biased manner to eliminate the obstruction without altering the opposite side of the septal wall. Thus, as shown in FIG. 2, the smaller electrode 230 is advanced to the side having the obstruction, while the larger electrode is advanced to the opposite side. If a more localized ablation is required, the difference in size of the electrodes may be increased, or is a deeper ablation is required the difference in size may be decreased. Because the electrodes have a variable size, a physician can adjust the ablation depth during a procedure.

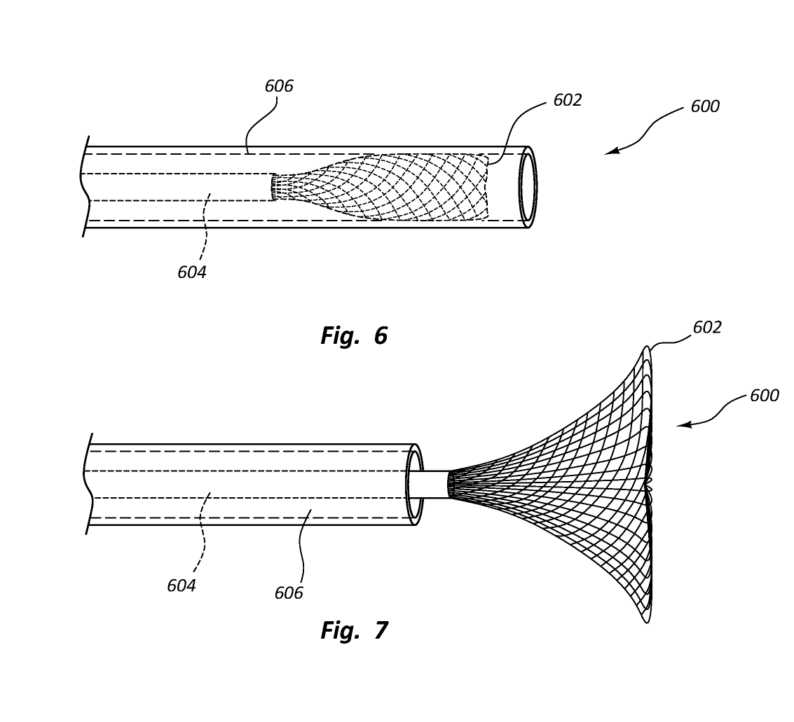

FIG. 6 illustrates another embodiment of a catheter assembly 600 having an expandable electrode 602 suitable for use with the present invention. The catheter assembly 600 has an inner catheter 604 and an outer catheter 606. The expandable electrode 602 is self-biased to an expanded configuration shown in FIG. 7. The outer catheter 606 is slidable relative to the inner catheter 604. In a first position shown in FIG. 6, the outer catheter 606 covers the expandable electrode 602 constraining it to the collapsed configuration shown in FIG. 6. The outer catheter 606 is slidable to a second position shown in FIG. 7, in which the expandable electrode 602 expands. The outer catheter 606 may be positioned at a position between the first position and the second position to vary the amount that the expandable electrode 602 expands.

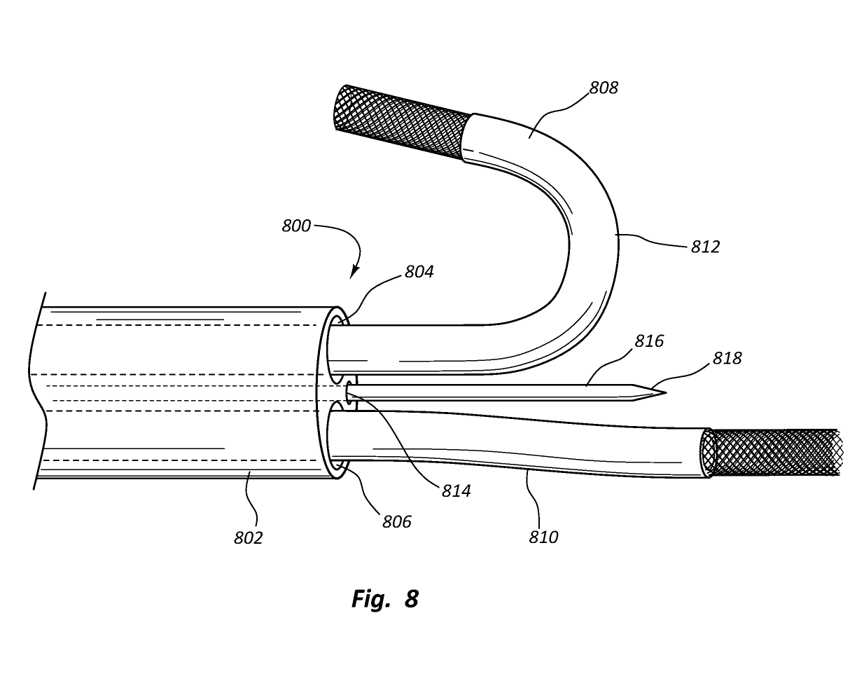

FIG. 8 illustrates the distal end of an embodiment of a bipolar ablation device 800 having a first catheter 802 having a first lumen 804 and a second lumen 806. A second catheter 808 such as the catheter assembly of FIG. 3 is disposed within the first lumen 804 and a third catheter 810 such as the catheter assembly of FIG. 3 is disposed within the second lumen 806. The bipolar ablation device 800 may be guided near the treatment area such as the basal septum and then the second catheter 808 and the third catheter 810 can be individually extended from the first lumen 804 and second lumen 806 and guided to the treatment site. In some embodiments, the second catheter 808 may be self-biased to a U shape 812 as shown in FIG. 8 such that when extended from the first lumen 804, the second catheter 808 curves back towards the first catheter 802.

The first catheter 802 may have a third lumen 814 with a puncture tool 816 disposed therein. The puncture tool 816 comprises an elongated body with a sharp point 818. In use, the first catheter 802 is guided to a location near the treatment site. The puncture tool 818 is then advanced from the third lumen 814 with force sufficient to pierce a septum. The puncture tool 818 may then be retracted into the third lumen 814. The second catheter 808 may then be advanced through the puncture and the U-shaped self-bias guides an electrode at the tip of the second catheter 808 to an opposite side of the septum.

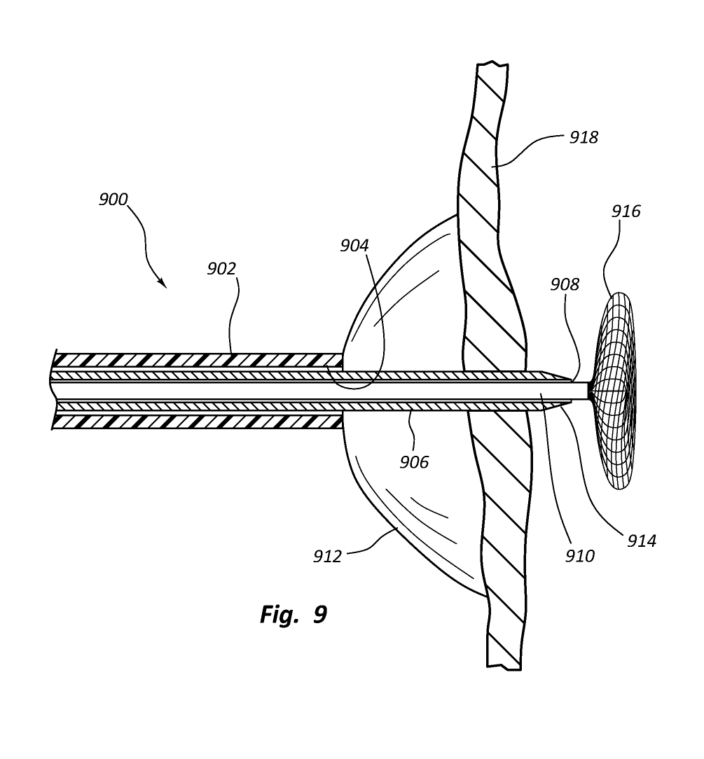

FIG. 9 illustrates another embodiment of a bipolar ablation device 900. The bipolar ablation device comprises a first catheter assembly 902 having a first lumen 904, a second catheter 906 having a second lumen 908, and a third catheter assembly 910. The first catheter assembly 902 has an expandable electrode 912 disposed at a distal end of the first catheter. The first catheter assembly 902 and the expandable electrode 912 may be of the type described in relation to FIG. 3 and FIG. 6 and will not be described in more detail here. The second catheter 906 is disposed in the first lumen 904. The second catheter 906 functions as a puncture tool and has a sharpened point 914. The third catheter assembly 908 is disposed within the second lumen 908 and has an expandable electrode 916 disposed at the distal end of the third catheter assembly 908. The third catheter assembly 908 and the expandable electrode 916 may be of the type described in relation to FIG. 3 and FIG. 6 and will not be described in more detail here.

In operation, the bipolar ablation device 900 is guided to a septum 918 requiring treatment. The second catheter 906 is extended from the first catheter assembly 902 and punctures the septum 918. The third catheter assembly 908 is then extended from the second catheter 906 and the expandable electrode 916 is expanded. The third catheter assembly 908 and/or the second catheter 906 may then be retracted until the expandable electrode 916 contacts the back of the septum 918. The expandable electrode 912 of the first catheter assembly 902 is expanded to a desired size and advanced to the front of the septum 918. With the electrodes on both sides of the septum 918, power is applied to the two electrodes and the tissue is ablated between them.

The above Figures and disclosure are intended to be illustrative and not exhaustive. This description will suggest many variations and alternatives to one of ordinary skill in the art. For example, while the illustrated embodiments are shown with cylindrical meshes, embodiments of the invention are not limited to such. The meshes may be formed in other shapes such as a rectangular tube or polygonal tube. It is contemplated that the different described embodiments may be combined with one another. All such variations and alternatives are intended to be encompassed within the scope of the attached claims. Those familiar with the art may recognize other equivalents to the specific embodiments described herein which equivalents are also intended to be encompassed by the attached claims.

What is claimed:

1. A bipolar ablation device comprising:
a first elongated member having a first distal end and a second proximal end, a first electrode disposed at the first distal end of the first elongated member and in electrical communication with the first proximal end of the first elongated member, the first electrode having a first size; and
a second elongated member having a second distal end and a second proximal end, a second electrode disposed at the second distal end of the second elongated member and in electrical communication with the second proximal end of the second elongated member, the second electrode being expandable from a collapsed configuration to an expanded configuration having a second size larger than the first size.

2. The bipolar ablation device of claim 1 further comprising an expandable mesh disposed at the second distal end, the expandable mesh being expandable from a collapsed mesh condition to an expanded mesh condition, wherein the second electrode is disposed on the expandable mesh.

3. The bipolar ablation device of claim 2 wherein the second electrode comprises a flexible conductive ink printed on the expandable mesh.

4. The bipolar ablation device of claim 3 wherein the expandable mesh is self-biased to the expanded configuration and the bipolar ablation device further comprises a sheath disposed about the second distal end, wherein the sheath is configured to slide between a first position axially adjacent the expandable mesh and a second position covering the mesh.

5. The bipolar ablation device of claim 3 wherein the expandable mesh is self-biased to the collapsed configuration and the bipolar ablation device further comprises a compression mechanism adapted to axially compress the expandable mesh from the collapsed configuration to the expanded configuration.

6. The bipolar ablation device of claim 1 further comprising a third elongated member having at least 2 lumens, wherein the first elongated member is disposed in the first lumen and the second elongated member is disposed in the second lumen.

7. The bipolar ablation device of claim 6 wherein the first electrode comprises a second expandable mesh, and wherein the first electrode is disposed on a proximal end of the expandable mesh.

8. The bipolar ablation device of claim 6 wherein the first elongated member is self-biased to a U shape at a distal portion of the first elongated member.

9. The bipolar ablation device of claim 1 wherein the second electrode comprises a plurality of point electrodes.

10. A method of ablating a septum utilizing a bipolar ablation device having a first electrode in electrical communication with a first pole of a power source and a second electrode in electrical communication with a second pole of a power source, the method comprising:
guiding the first electrode to an ablation target area on a first side of a septum, the first electrode having a first size;
guiding the second electrode to an area on a second side of the septum;
expanding the second electrode to an expanded configuration having a second size greater than the first size; and
providing power from the power source to the first electrode and the second electrode to ablate tissue at the target area.

11. The method of claim 10 wherein the ablation device further comprises a sheath covering the second electrode, wherein the second electrode is self-biased to the expanded configuration, and wherein expanding the second electrode comprises extending the second electrode from the second sheath.

12. The method of claim 10 further comprising puncturing the septum and guiding the first electrode through the puncture.

13. The method of claim 10 further wherein the bipolar ablation device further comprises an inner catheter and an outer catheter, wherein the second electrode is a flexible mesh covered with a conductive coating, the second electrode having a distal end coupled to the inner catheter and a proximal end coupled to the outer catheter, wherein expanding the second electrode comprises extending the inner catheter from the outer catheter.

14. A bipolar ablation device comprising:
a first ablation assembly comprising;
a first outer catheter having a first outer distal end and a first outer proximal end;
a first inner catheter having a first inner distal end and a first inner proximal end, the first inner catheter being slidably disposed within a lumen of the first outer catheter;
a first flexible mesh having a first flexible mesh proximal end coupled to the first outer distal end and a first flexible mesh distal end coupled to the first inner distal;
a first flexible conductive coating disposed on the first flexible mesh; and
a conductor configured to couple the first flexible conductive coating to a first pole of a power source; and
a second ablation assembly comprising;
a second outer catheter having a second outer distal end and a second outer proximal end;
a second inner catheter having a second inner distal end and a second inner proximal end, the second inner catheter being slidably disposed within a lumen of the second outer catheter;
a second flexible mesh having a second flexible mesh proximal end coupled to the second outer distal end and a second flexible mesh distal end coupled to the second inner distal;
a second flexible conductive coating disposed on the second flexible mesh; and
a conductor configured to couple the second flexible conductive coating to a second pole of a power source.

15. The bipolar ablation device of claim 14 wherein the first flexible mesh is stored within the lumen of the first outer catheter and the second flexible mesh is stored within the lumen of the second outer catheter.

16. The bipolar ablation device of claim 14 further comprising a delivery catheter having a first lumen and a second lumen, wherein the first ablation assembly is disposed in the first lumen and the second ablation assembly is disposed in the second lumen.

17. The bipolar ablation device of claim 16 wherein the second ablation assembly is self-biased to form a U shaped curve at a distal end of the second ablation assembly.

18. The bipolar ablation device of claim 16 wherein the second ablation device is steerable remotely.

19. The bipolar ablation device of claim 16 wherein the delivery catheter has a third lumen, and wherein the septum ablation device further comprises a puncture tool disposed in the third lumen.

20. The bipolar ablation devise of claim 14 further comprising a puncture tool disposed within a lumen of the first inner catheter, wherein the second catheter assembly is disposed within the lumen of the first ablation assembly, and wherein the puncture tool and second catheter assembly are extendable from the lumen of the first ablation assembly.
